(19) United States
(12) Patent Application Publication
(10) Pub. No.: US 2008/0078414 A1
(43) Pub. Date: Apr. 3, 2008

(54) Lip sealer device and methodology

(76) Inventor: Nickolas P. Demas, Neshanic Station, NJ (US)

Correspondence Address: Kenneth P. Glynn, 24 Mine Street, Flemington, NJ, 08822, US

(21) Appl. No.: 11/521,249

(22) Filed: Sep. 14, 2006

(51) Int. Cl. A61F 11/00

(52) U.S. Cl. 128/857

(57) ABSTRACT

A methodology and device for inhibiting or reducing dry mouth and inhibiting or reducing snoring of a human during sleep consists of applying a lip sealer device to a human in an angular position on the head. The lip sealer device forms a flexible, elastic continuous loop. The device may be positioned in the front of the head above the chin and below the lower lip, and extends diagonally across the cheeks and over the top of each ear and across the back of the head. The device has holding force to inhibit the opening of the lips during sleep. Optional lip balm may be used to seal the lips during sleep.

### BACKGROUND OF INVENTION

a. Field of Invention

The invention relates generally to solving some of the problems that occur during sleeping hours for many people, namely, snoring and dry mouth. Thus, the present invention is a device and a methodology for keeping the mouth shut and the lips closed of a sleeper who would otherwise experience the foregoing problems. The present invention involves applying a lip sealer device to a human about the head in a specified manner.

b. Description of Related Art

The following patents are representative of various devices and methods of holding a mouth or jaw closed:

U.S. Pat. No. 7,063,084 B2 to McDonald describes a support for an oxygen diffuser for delivering a plume of oxygen-enriched air to a space in front of a patient's nose and mouth. The support comprises a means for holding a tube associated with the oxygen diffuser. This means is located at the vertex of front portions of rigid arms forming a v-shaped front of the support. A resilient front strap extends between rear portions of the arms. A resilient back strap extends between ends of the rear portions of the arms. In operation, the back strap is arranged so as to extend behind a user's head and is releasably adjustable to an operative length to seat the support securely on a user's head with a portion of the front of the user's face bearing against the front strap, so that, when so seated, the diffuser is held in a position spaced from but proximal to the patient's nose and mouth.

U.S. Pat. No. 6,802,079 B2 to Bush describes a mouth protector assembly comprising a grid-like mouth protector, defined by a top bar contoured to engage the area above the upper lip and below the nose of a user, a series of spaced bars depending from the upper bar and a pad which underlies the chin of a user, a strap system for supporting the grid over the mouth including straps connected to the grid and encircling the head to support the grid over the mouth of the user.

U.S. Pat. No. 6,148,820 to Herrin describes an apparatus for inhibiting nocturnal drying of the oral cavity from breathing through a person's mouth. More particularly, the invention is directed to an apparatus comprising an air permeable cover, an elastic strap attached to the cover for securing the cover to a person's face, and a cup attached to the lower region of the cover for securing a person's chin.

U.S. Pat. No. 5,787,894 to Holt describes a new Jaw-Closing Anti-Snoring System for controlling a user's snoring by supporting the user's jaw in a closed position while sleeping. The inventive device includes a strap having a first end and a second end, a securing means at the first and second end allowing the first end to removably secure to the second end, and an arcuate jaw support member formed to the shape of a jaw secured to the concentric portion of the strap and supporting the jaw of the user.

U.S. Pat. No. 5,785,052 to Johnson describes a versatile disposable film protective mask which includes an outlined boundary, a top pair of primary film strings, two top pairs of secondary film strings/ear loops, a bottom pair of primary film strings, a well-defined chin guard having chin guard receptors, jaw webs, cheek webs, an oral cavity web having oral cavity receptors and a reversible half-oval shaped flaccid oral cavity pouch. In a preferred embodiment, the versatile disposable film protective mask further includes a thin lining for those who are allergic to the film and to absorb liquids, i.e. saliva and/or perspiration produced by the wearer of the mask. The versatile disposable film protective mask can also be employed as a resuscitation mask, a dental dam, a male G-string flaccid pouch with a disposable film flaccid pouch harness or a female G-string flaccid pouch with disposable film flaccid pouch harness. The versatile disposable film protective mask is employable for protecting the wearer against oral and genital infections with communicable diseases including viral diseases such as Human Immunodeficiency Virus (HIV) which has been known to lead to the development of Acquired Immunodeficiency Syndrome (AIDS). The mask is also employable for protecting the wearer against other oral and genital infections and Sexually Transmitted Diseases (STD's).

U.S. Pat. No. 5,687,743 to Goodwin describes a head strap assembly for reducing snoring activity including a front strap for extending around a head spaced forwardly of ears and below a chin of a user, a rear strap for extending around the head and rearwardly of the ears and above the chin of the user with the rear strap crossing the front strap between the ears and chin of the user, and a link strap interconnecting the front and rear straps above the ears of the user. The front strap preferably has an upper portion and a lower portion with pairs of adjacent ends for positioning on each side of the head of the user. The assembly may also include adjustable fastening elements which are attached to the pair of adjacent ends of the upper and lower portions of the front strap for providing overlapping releasable coupling of the adjacent ends of the upper and lower portions of the front strap to one another. The assembly may also include an upper chin pad, a lower chin pad and a head pad. The upper chin pad is slidably mounted to the rear strap for positioning above the chin of the user. The lower chin pad is slidably mounted to the lower portion of the front strap for positioning below the chin of the user. The head pad is slidably mounted to the link strap for positioning on the head of the user.

U.S. Pat. No. 5,669,377 to Fenn describes a user friendly nasal band and method which lifts up the tip of the nose, enlarges the nostrils and shortens the effective length of the nasal passageways. The light weight nasal band provides a comfortable sleep band and dilator strip which improves sleep and makes breathing easier for many patients. Advantageously, the sleep band and method also reduces or eliminates snoring for many patients. This is accomplished by improving the nasal airway. An improved nasal airway minimizes mouth breathing which prevents vibration of the palate, tongue, and pharyngeal walls thus reducing the noise of snoring. Air passing though the mouth during sleep causes vibration of the relaxed tongue, palate, and pharyngeal wall resulting in the noise of snoring. The nasal band also provides an attractive sports band which minimizes collapse of the nasal sidewalls during jogging or other strenuous exercise so that athletes can continue to breathe through their nose rather than breathe through their open mouth. The nasal band is preferably worn vertically and stretches along the bridge of the nose from the tip of the nose to the upper part of the nasal bridge or forehead. The nasal band can comprise a waterproof adhesive tape with a lower tip portion, a bridge portion, and a top portion.

U.S. Pat. No. 5,546,929 to Muchin describes a nasal dilator for preventing outer wall tissue of nasal passages of a nose from drawing in during breathing. There is a spring member for bridging a human nose, the spring member extending over the bridge and at least partly beyond the bridge on both sides of the bridge. A pad with an adhesive surface covers the spring member and extends around the spring member so that there is a perimeter of space formed between the spring member and the pad member. The spring is inset centrally in the pad. An adhesive between the spring member and the pad wholly connects the spring member on its entire engaging surface with the pad.

U.S. Pat. No. 5,533,504 to Stamos describes an apparatus for controlling air flow through nasal passages having a frame with first and second plates for positioning on a face along opposite sides of a nose. The plates have first and second extensions extending forwardly and inwardly and contacting a face of the user beneath the nose and above an upper lip. -Rods extend inward from the plates, and nose-engaging pads are mounted on inward proximal ends of the rods. A strap connects the plates and extends rearwardly around a head of a user. The strap has first and second sections with hook and loop complementary fasteners for adjusting the strap to fit a head of a user, and holding the strap. The extensions have a curved bar which slides within a curved channel for adjusting the position between the plates. A threaded clamp has an enlarged head mounted in an elongated recess at the rear of the channel, and has a shaft which extends through an elongated opening in the channel and through an opening in the extension which slides within the channel. A fastener nut is threaded on an outer end of the fastener shaft to clamp the extensions in a selected position. Inward facing surfaces of the plates have cushions along the rear edge and bottom edge portions, and the channel extension has cushions along the edges for contacting the user's face. The plates have multiple threaded openings in which the threaded rods may be selectively received.

U.S. Pat. No. 5,361,416 to Petrie et al. describes a headcover and chin cup assembly which maintains a closed mouth during the treatment of sleep apnea and makes the user breathe only through his or her nose. The assembly is made up of a headcover which fits easily and securely on the head of practically all people. The headcover and the chin cup each include a set of straps and a set of D-rings. The set of straps attached to the headcover engage the set of D-rings attached to the chin cup. The set of straps attached to the chin cup engage the set of D-rings attached to the headcover. Each set of straps is adjusted secured via fasteners. The chin cup holds the apex of the user's chin for a secure fit.

U.S. Pat. No. 5,016,649 to Johnson describes an invention which relates to a protective mask, and more particularly to a protective mask usable during the act of oral sex or during the performance of mouth-to-mouth resuscitation to protect a wearer against infection with communicable diseases including viral diseases such as the human immune virus which leads to the development of acquired immune deficiency syndrome (AIDS). The mask is formed by an elongate, generally planar membrane of tough film material having, when lying flat, a longitudinal axis and an outline configuration which is asymmetrical about that axis, the outline configuration having a lateral extension portion for extending downwardly and rearwardly over the chin of a wearer. The mask is securely wrapped in place over the lower face of a wearer while permitting free movement of the jaw, lips and tongue of the wearer and includes a flaccid pouch-like central portion.

U.S. Pat. No. 4,934,357 to Frantzich et al. describes ajaw support body comprised of ajaw and chin engaging portion and cheek and temporal engaging portions. First and second pairs of mating straps extend from the jaw support body, and each strap is provided with a quick release type fastener on its outer end for attachment to its associated mating strap. When the jaw support bandage is applied to the head of a patient, the mating interconnected straps are spaced from each other a predetermined distance to prevent slippage of the bandage from the patient's head. In one embodiment of the invention, the mating interconnected straps extend over the coronal portion of the patient's head. In another embodiment of the invention, one mating pair of straps extends over the coronal portion of the patient's head and the other mating pair of straps extends around the forehead and rear portion of the patient's head.

U.S. Pat. No. 3,480,010 to R. B. Crossley describes an anti-snore device consisting of a neckband to be worn by a sleeper. The neckband contains a microphone, a transistor amplifier, a high voltage-producing transformer, a supply battery, a relay and a pair of spaced electrodes. The electrodes are in contact with the sleeper's skin. The microphone is connected to the input of the amplifier and the relay winding is connected to the output of the amplifier. The primary of the high voltage-producing transformer is connected in series with the relay contacts and the supply battery. The amplifier is energized from the supply battery. Snore is detected by the microphone to produce output signal waves of corresponding pitch, causing intermittent closure at this frequency of the relay contacts, thereby inducing a high voltage across the electrodes designed to shock the sleeper and to condition him against snoring.

U.S. Pat. No. 2,507,617 to G. A. Swendiman describes ajaw supporting apparatus, a central member pre-shaped to snugly fit the chin. The central member has aligned V-shaped pockets, and side members that include relatively wide wing portions for engaging the sides of the jaw and tapering strap portions. The tapering strap portions are adapted to extend into the pockets. When the straps are fastened into the pockets, a plurality of spaced strap fastening elements form at the lower portion of the central member below the pockets. A pair of super-imposed fastening elements extend outwardly from the wings. A supporting harness includes a head band, and strap members connect the head band and the fastening elements for supporting the central member and the jaw side members.

U.S. Pat. No. 1,990,411 to O. Lowry describes a device for preventing mouth breathing and snoring, comprising an elastic strap for disposition around the head of the user in the region of the forehead. Also, it comprises a second strap disposed in crossing relation with the first-mentioned strap and having a running engagement with the latter at the points of crossing. It is adapted to extend over the plate of heart-shaped formation presenting the same with a broadened recessed rear edge portion. The latter-mentioned portion is disposed in spaced confronting relation with respect to the throat of the user. The recess is formed in the broadened edge of the heart-shaped chin rest, which is concaved, thereby affording clearance of the chin rest plate with the throat. A loop formed integral with the under face of the chin rest plate and extending transversely thereof to accommodate the mounting and adjustment of the plate on the second-mentioned strap. The chin rest plate is provided with a narrow forward end and a concaved upper face portion.

U.S. Pat. No. 1,339,865 to J. W. Rothenberger describes an anti-mouth breathing device comprising a chin plate adapted to receive the chin of the patient. The chin plate has a forwardly extending flange projecting outwardly from the chin. The side bars extend rearwardly from the opposite ends of the plates and vertically disposed to lie close to the wearer's jaws. Means for retaining the plates in position comprise a head band and a pair of transversely disposed straps connected to the head band at their extremities and at either side of the head. They also comprise a pair of forward supporting straps connected to the band adjacent to one of the transverse straps and to the flange; a pair of rear straps connected to the band; side bars in front of the ears; and divergent straps also connected to the bars at the ends thereof and to the band.

U.S. Pat. No. 1,296,946 to E. V. Galiardo describes an apparatus for controlling respiration and teaching breathing through the nose. It comprises the combination of a mask adapted to press upward on the lower jaw and head straps passing around the head of the wearer. The head straps are adapted to exert a yielding upward pressure on the mask sufficient to gently hold the lower jaw closed, but permitting the jaw to be easily opened when desired. Retaining means are carried by the mask for positioning a medicinal substance in the path of the breath inhaled through the nose and a medicinal substance in the retaining means adapted to clear the air passages through the nose and head and encourage breathing therethrough.

U.S. Pat. No. 1,216,679 to G. A. Foster describes a device for preventing snoring, having a combination of textile means encircling the chin and covering the mouth of the wearer, having an imperforate rubber lining on the mouth covering portion only of the means to firmly hold the lips in closed position; also having adjustable straps for securing the device in operative position.

U.S. Pat. No. 1,134,993 to M. Bye describes a medicament container having a hollow rectangular holder open at one end and having a solid cake of medicinal material inserted into the holder at the open end. The inner end of the cake is projected into the interior of the holder and forms a base for a supply of lumps of medicinal material. The holder has a closed wall at the other end provided with a pair of nostril engaging openings spaced apart longitudinally of the end wall. The holder has apertures in its side walls between the end of the cake and the closing wall for the purpose of admitting air to the lumps of material.

U.S. Pat. No. 1,043,924 to M. E. G. Gottlieb describes an apparatus of the kind described as the combination of a central structure adapted to be applied to the nose and two arms. The means for pivotally connecting the arms with the central structure are springs secured to the arms and adapted to bear with their ends on inclined parts of the central structure. Heads at the free ends of the arms are means for securing the apparatus to the head, substantially as and for the purpose set forth.

U.S. Pat. No. 649,896 to J. S. Baughman describes a bandage of the character set forth, having a sling to fit over the chin, comprising upper and lower members connected by an intermediate web in a plane at right angles to the members. The lower member is longer than and has its extremities attached to the extremities of the upper member to thereby give the proper shape to the sling and position the upper member under the lower lip. When applied, elastic straps attach to the united ends of the members and have means for holding the elastic straps in place.

Notwithstanding the prior art, the present invention is neither taught nor rendered obvious thereby.

### SUMMARY OF INVENTION

The invention solves the problems and overcomes the drawbacks and deficiencies of prior art methods and devices for attempting the keep a mouth closed to prevent or inhibit snoring and/or dry mouth, by providing a method and device that attaches above the chin instead of below it to effectively keep the lips as well as the mouth closed.

The present invention is a methodology and device for inhibiting or reducing dry mouth and inhibiting or reducing snoring of a human during sleep, which consists of applying a lip sealer device to a human in an angular position on the head. The lip sealer device forms a flexible, elastic continuous loop. The device may be positioned in the front of the head above the chin and below the lower lip, and extends diagonally across the cheeks and over the top of each ear and across the back of the head. The device has holding force to inhibit the opening of the lips during sleep.

In some preferred embodiments, the methodology for inhibiting or reducing dry mouth and inhibiting or reducing snoring of a human during sleep lip sealer device may be a flexible, elastic continuous loop that has length adjustment means. The length adjustment means may be a buckle or a clip with the flexible, elastic continuous loop having a first terminus and a second terminus wherein the buckle or clip is located proximate one of the termini and the other of the termini may be positioned through the buckle or clip to create a continuous loop.

In some preferred embodiments, the device may be a single elastomer band and an adjustment clip, with each of the termini being looped through the adjustment clip. The adjustment clip may be a single component with four orifices.

In some preferred embodiments, the flexible, elastic continuous loop is a biocompatible elastomer that may be selected from the group consisting of a buna-s rubber material, an oil resistant neoprene material, an oil resistant silicone material, an oil resistant buna-n rubber, or any other oil resistant elastomer. In some embodiments, the continuous loop may include a pair of ear mounts that broadly encircle the ears of the human. The continuous loop may include a front flexible linear member, a rear flexible linear member, a pair of flexible ear mounts and means for connecting each of the ear mounts to opposite ends of each of the front flexible linear member and rear flexible linear member.

The present invention also includes the methodology for inhibiting or reducing dry mouth and inhibiting or reducing snoring of a human during sleep, which includes utilizing the above described device and applying a lip balm to the lips of a human to create marginal adherence of the lips to one another. In preferred embodiments, the entire mouth opening area between the lips will be coated on the lips to create an air tight seal. The lip sealer device may be a flexible elastic continuous loop having length adjustment means of the aforesaid types.

The length adjustment means may be a buckle and the flexible continuous loop may have a first terminus and a second terminus wherein the buckle may be located proximate one of the termini and the other of the termini positioned through the buckle to create the continuous loop.

The device may be a single elastomer band and adjustment clip, with each of the termini being looped through the adjustment clip. The adjustment clip may be a single component with four orifices.

The flexible, elastic continuous loop may be a material selected from the group consisting of rubber, buna-s rubber and other elastomers as described above. It includes a pair of ear mounts that broadly encircle the ears of the human, a front flexible linear member, a rear flexible linear member, a pair of flexible ear mounts and connecting means for permanently connecting each of the ear mounts to opposite ends of each of the front flexible linear member and rear flexible linear member.

The device for inhibiting or reducing dry mouth and inhibiting or reducing snoring of a human during sleep may be a continuous loop that includes a front flexible linear member, a rear flexible linear member, a pair of flexible ear mounts and means for connecting each of the ear mounts to opposite end of each of the front flexible linear member and rear flexible linear member. The connecting means may be four short sealed loops, one each located at opposite ends of the front flexible linear member and the rear flexible linear member. The ear mounts may be continuous loops adapted to encompass a human ear.

The flexible continuous loop may be a buna-s rubber material. The connecting means may be four short sealed loops, one each located at opposite ends of the front flexible linear member and the rear flexible linear member. The ear mounts may be continuous loops adapted to encompass a human ear.

Additional features, advantages, and embodiments of the invention may be set forth or apparent from consideration of the following detailed description, drawings, and claims. Moreover, it may be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a side view of an embodiment of a lip sealer device according to the present invention, illustrating it in use;

FIG. 2 is a front view of the present invention lip sealer device of FIG. 1, illustrating the frontal position of the device;

FIG. 3 is a block diagram of the present invention methodology;

FIG. 4 is a front view of an alternative present invention lip sealer device with ear mounts;

FIG. 5 is a side view of the present invention device shown in FIG. 4;

FIG. 6 is a front perspective view of the present invention device shown in FIGS. 4 and 5 in its unused, unstretched state; and,

FIG. 7 and FIG. 8 show top and side views of another embodiment of a lip sealer device according to the present invention.

### DETAILED DESCRIPTION OF THE EMBODIMENTS

Referring now to the drawings, like reference numerals designate corresponding parts throughout the several views, for each embodiment. Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

The accompanying drawings, which are included to provide a further understanding of the invention and are incorporated in and constitute a part of this specification, illustrate preferred embodiments of the invention and together with the detail description serve to explain the principles of the invention. In the drawings:

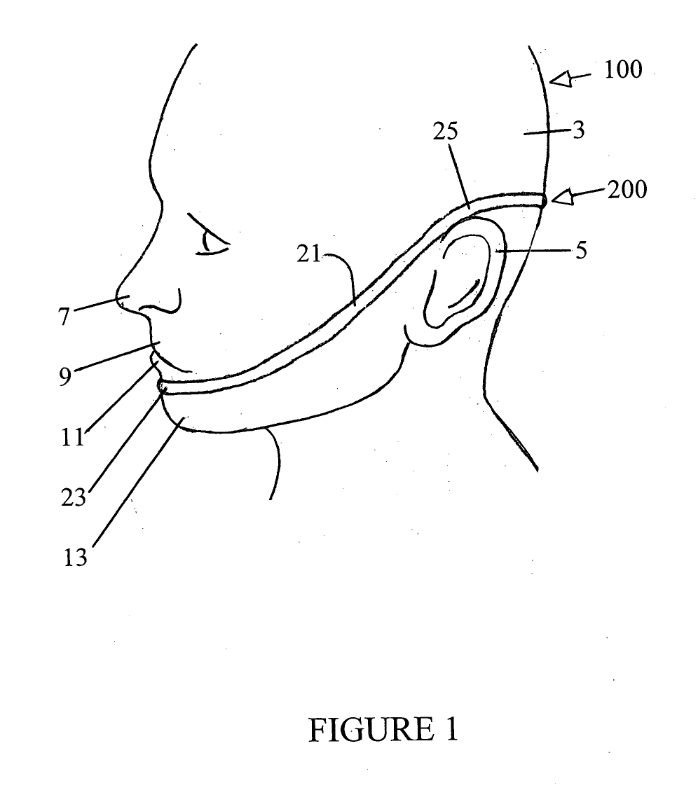

FIG. 1 is a side view of a preferred embodiment of a lip sealer device 200 according to the present invention, illustrating it in use on a human 100. Human 100 is in a sitting or standing awake position for illustrating the present invention, but once the device 200 has been applied it is expected that the human 100 will lay down and sleep immediately or shortly thereafter. Device 200 is a continuous loop of flexible, stretchable (elastic) material that is preferably elastomer of the buna-s rubber type. As can be seen in FIG. 1, device 200 is applied at an angular position at the head (meaning not perfectly vertical or perfectly horizontal) with the continuous loop positioned in the front of head 3 so as to be completely away from nose 7 and below upper lip 9 and lower lip 11 while above chin 13. In other words, device 200 has a front portion 23 that is located in the small area bellow both lips and above the chin. Unlike other devices that are positioned on or below the chin, this position not only pushes up the chin and jaw but also biases the lower lip towards and against the upper lip. Portion 21 of present invention 200 passes generally across the middle of the cheek and upon the tops of the ears and around the back of the head 3. Thus, portion 25 is located above ear 5 and then traverses the back of the head at about the same level. Different positionings will result in slippage up toward, against, or over lip 11, while lower positions will fail to push the lips closed.

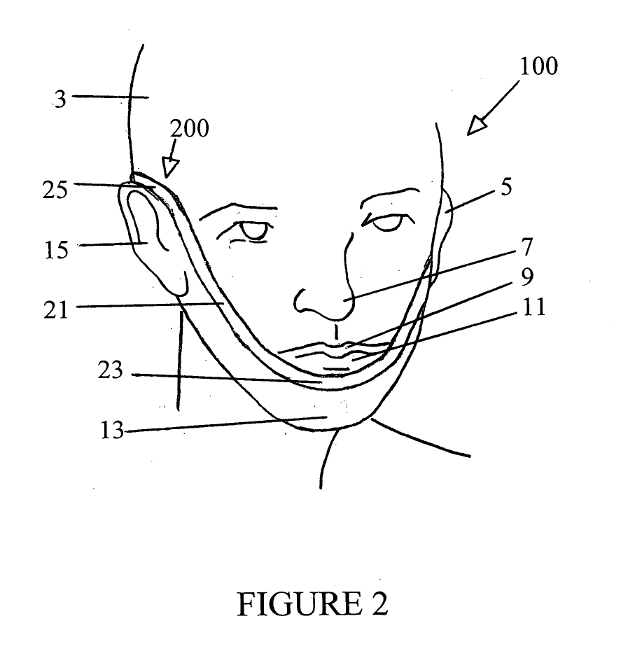

FIG. 2 is a front view of the present invention lip sealer device of FIG. 1, illustrating the frontal position of the device. Here, both ear 5 and ear 15 are shown and the symmetrical arrangement of present invention device 200 can be appreciated. Identical parts are other wise identically numbered as in FIG. 1. It is the unique positioning of device 200 and its material composition that effectively keeps the mouths and the lips 9 and 11 of human 100 closed during sleep. Optional lip balm, such as petrolatum-based products (petroleum jelly) and wax-based products, may be applied to lips 9 and 11 to keep them from unintentionally opening slightly in sleep, i.e. seals the lips and eliminates leakage of air. The adherent stickiness of these products is an optional supplement step.

The flexible, elastic continuous loop present invention device 200 was found to be unexpectedly effective and efficient when made of buna-s rubber. When of other materials were used, rolling or slipping of the device occurred. Thus, silicone devices, neoprene rubber device, when tested, were not as effective or efficient as the buna-s rubber products. The latex products sometimes caused allergic reactions. The silicon and neoprene products deteriorated and lost their staying capabilities from body oils as well as from applied facial and skin creams. Hence, oil resistant types of these materials are preferred. Additionally, rolling and slipping may be further inhibited by the shape of the product. Flat surfaces are preferred over round or curved surfaces.

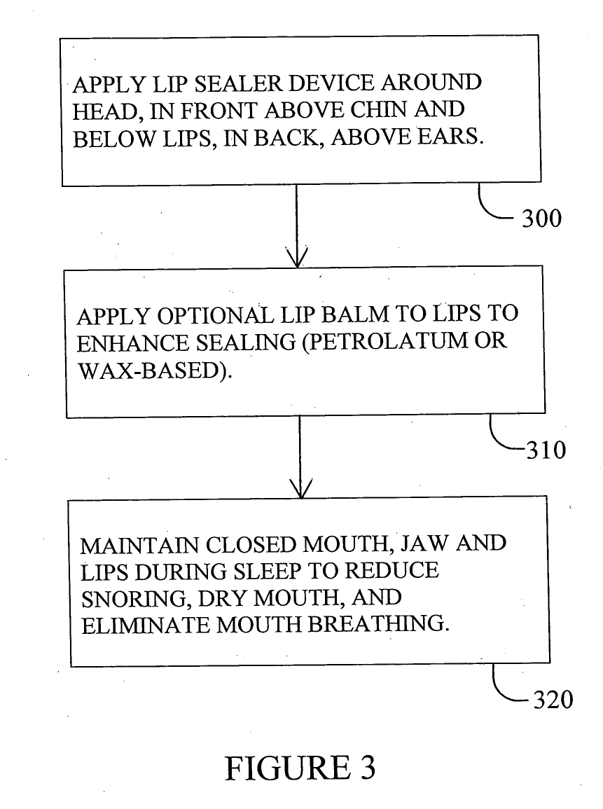

FIG. 3 is a block diagram of the present invention methodology. Block 300 involves a step of applying the present invention device to the head in front above the chin and below the lips and above the ears. Block 310 relates to optional application lip balm to the lips. Block 320 illustrates maintaining advantageous mouth jaw and lip positioning during sleep with reduction in snoring and dry mouth.

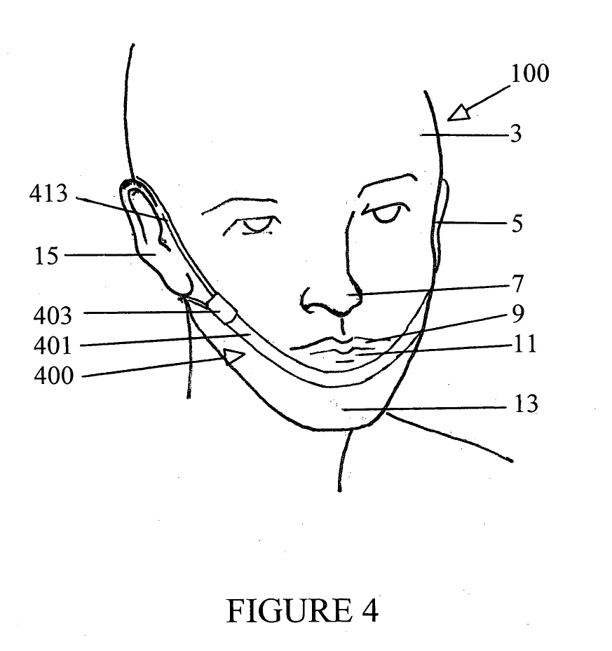

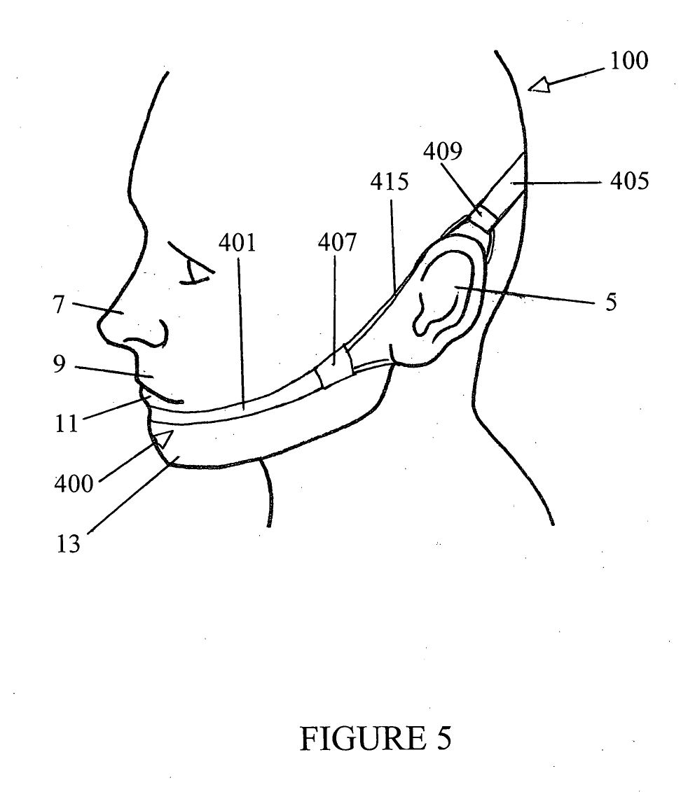

FIG. 4 is a front view of an alternative present invention lip sealer device with ear mounts. The human 100 is the same person with identically numbered parts as in FIGS. 1 and 2. In this embodiment, present invention device 400 is a continuous loop formed of four different components. FIG. 5 is a side view of the present invention device shown in FIG. 4. These two FIGS. 4 and 5 are now described collectively. Device 400 has a front flexible, stretchable, linear member 401 with ear mount attachment means 403 and 407 separate ear mount loops for 413 and for 415 and a rear flexible, stretchable, linear member 405. With attachment means 409. The upper part of the ear mount loops are positioned above the ears and this feature is important for pulling lower lip 11 toward upper lip 9. Thus, device 400 functions in a manner similar to device 200 described except that ear mounts help the user to more easily position the device.

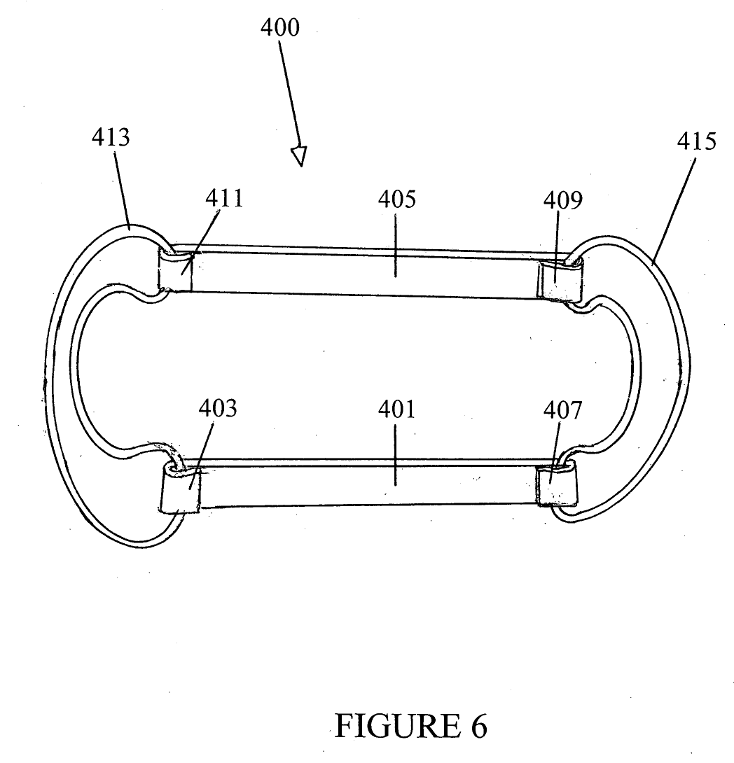

FIG. 6 is a perspective front view of the present invention device shown in FIGS. 4 and 5 in its unused, unstretched state. Identical components are numbered identically. The two ear loops mount loops are buna-s rubber bands and front member 401 and 405 have looped and sealed ends to create the connecting means such as means 411 to create permanent attachments and the thus, to form a perfect continuous loop.

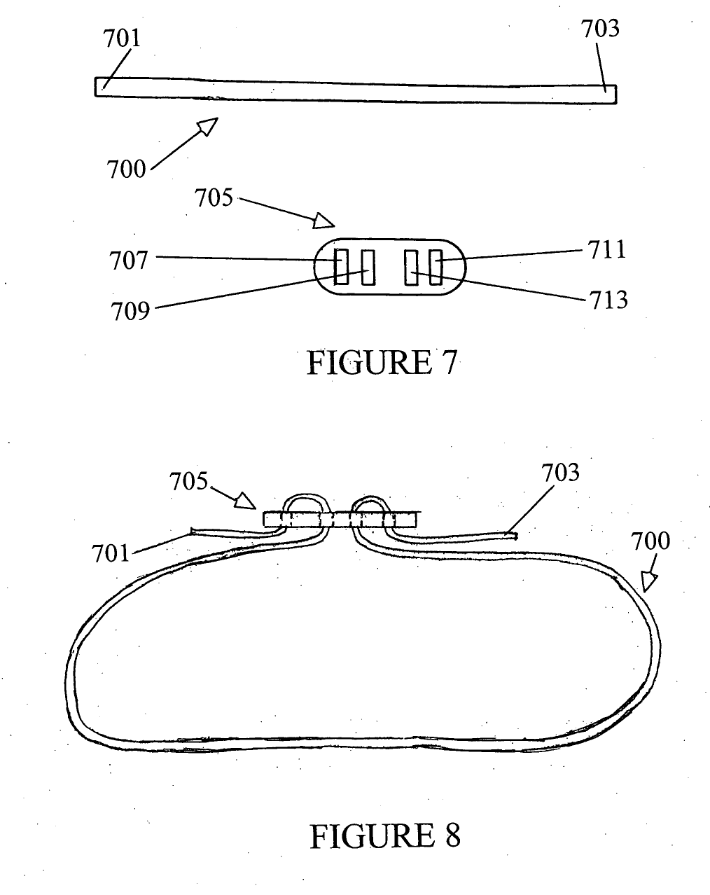

FIG. 7 and FIG. 8 show top and side views of a preferred embodiment of a lip sealer device according to the present invention. An elongated strap 700 is a flexible, stretchable member of sufficient length to wrap around an adult human head with surplus length. It is connected with attachment means buckle 705 as shown in FIG. 8 and applied to the head in accordance with the above Figure and instructions, and adjusted to the desired sized loop and tightness. Thus, end 701 is inserted into orifice 709 and back down orifice 707. Like wise end 703 is passed through orifice 713 and back down through orifice 711 and one or both ends are pulled to tighten or pushed to loosen. Other buckles, snaps and connecting members could be used with out exceeding the scope of the present invention.

Although particular embodiments of the invention have-been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

That is claimed is:

1. A methodology for inhibiting or reducing dry mouth and inhibiting or reducing snoring of a human during sleep, which consists of:
applying a lip sealer device to a human in an angular position on the head, said lip sealer device forming a flexible, elastic continuous loop, said device being positioned in the front of said head above the chin and below the lower lip, and extending diagonally across the cheeks and upon the top of each ear and across the back of said head, said device having holding force to inhibit the opening of the lips during sleep.

2. The methodology for inhibiting or reducing dry mouth and inhibiting or reducing snoring of a human during sleep of claim 1 wherein said lip sealer device is a flexible, elastic continuous loop having length adjustment means.

3. The methodology for inhibiting or reducing dry mouth and inhibiting or reducing snoring of a human during sleep of claim 2 wherein said length adjustment means is selected from the group consisting of a buckle and a clip, and said flexible, elastic continuous loop has a first terminus and a second terminus wherein said adjustable means is located proximate one of said termini and the other of said termini is positioned through said adjustable means to create said continuous loop.

4. The methodology for inhibiting or reducing dry mouth and inhibiting or reducing snoring of a human during sleep of claim 2 wherein said device is a single elastomer band and an adjustment clip, with each of said termini being looped through said adjustment clip.

5. The methodology for inhibiting or reducing dry mouth and inhibiting or reducing snoring of a human during sleep of claim 4 wherein said adjustment clip is a single component with four orifices.

6. The methodology for inhibiting or reducing dry mouth and inhibiting or reducing snoring of a human during sleep of claim 1 wherein said flexible continuous loop is a biocompatible elastomer selected from the group consisting of buna-s rubber, oil resistant neoprene, oil resistant silicone and oil resistant buna-n rubber.

7. The methodology for inhibiting or reducing dry mouth and inhibiting or reducing snoring of a human during sleep of claim 1 wherein said continuous loop includes a pair of ear mounts that broadly encircle the ears of said human.

8. The methodology for inhibiting or reducing dry mouth and inhibiting or reducing snoring of a human during sleep of claim 7 wherein said continuous loop includes a front flexible elastic linear member, a rear flexible elastic linear member, a pair of flexible elastic ear mounts and means for connecting each of said ear mounts to opposite ends of each of said front flexible linear member and rear flexible linear member.

9. A methodology for inhibiting or reducing dry mouth and inhibiting or reducing snoring of a human during sleep, which consists of:
applying a lip balm to the lips of a human to create marginal adherence of said lips to one another and to improve sealing of lips to one another.

10. The methodology for inhibiting or reducing dry mouth and inhibiting or reducing snoring of a human during sleep of claim 1 wherein said lip sealer device is a flexible elastic continuous loop having length adjustment means.

11. The methodology for inhibiting or reducing dry mouth and inhibiting or reducing snoring of a human during sleep of claim 2 wherein said length adjustment means is a buckle and said flexible continuous loop has a first terminus and a second terminus wherein said buckle is located proximate on of said termini and the other of said termini positioned through said buckle to create said continuous loop.

12. The methodology for inhibiting or reducing dry mouth and inhibiting or reducing snoring of a human during sleep of claim 2 wherein said device is a single elastomer band and adjustment clip, with each of said termini being looped through said adjustment clip.

13. The methodology for inhibiting or reducing dry mouth and inhibiting or reducing snoring of a human during sleep of claim 2 wherein said adjustment clip is a single component with four orifices.

14. The methodology for inhibiting or reducing dry mouth and inhibiting or reducing snoring of a human during sleep of claim 1 wherein said flexible elastic continuous loop oil resistant silicone.

15. The methodology for inhibiting or reducing dry mouth and inhibiting or reducing snoring of a human during sleep of claim 1 wherein said continuous loop includes a pair of ear mounts that broadly encircle the ears of said human.

16. The methodology for inhibiting or reducing dry mouth and inhibiting or reducing snoring of a human during sleep of claim 7 wherein said continuous loop includes a front flexible linear member, a rear flexible linear member, a pair of flexible ear mounts and connecting means for connecting each of said ear mounts to opposite ends of each of said front flexible linear member and rear flexible linear member.

17. A device for inhibiting or reducing dry mouth and inhibiting or reducing snoring of a human during sleep, which consists of:
a continuous loop that includes a front flexible, elastic linear member, a rear flexible, elastic linear member, a pair of flexible, elastic ear mounts and means for connecting each of said ear mounts to opposite end of each of said front flexible linear member and rear flexible linear member.

18. The device for inhibiting or reducing dry mouth and inhibiting or reducing snoring of a human during sleep of claim 17 wherein said flexible, elastic continuous loop is selected from the group consisting of buna-s rubber, oil resistant neoprene, oil resistant silicone and oil resistant buna-n rubber.

19. The device of claim 17 wherein said connecting means is four short sealed loops, one each located at opposite ends of said front flexible linear member and said rear flexible linear member.

20. The device of claim 17 wherein said ear mounts are continuous loops adapted to encompass a human ear.
